(19) United States
(12) Patent Application Publication
(10) Pub. No.: US 2023/0190192 A1
(43) Pub. Date: Jun. 22, 2023

(54) VITAL SENSOR INFORMATION COLLECTION SYSTEM, VITAL SENSOR INFORMATION COLLECTION METHOD AND VITAL SENSOR INFORMATION COLLECTION HUB TERMINAL

(71) Applicant: NIPPON TELEGRAPH AND TELEPHONE CORPORATION, Tokyo (JP)

(72) Inventors: Ryota SHIINA, Musashino-shi, Tokyo (JP); Yuki SAKAUE, Musashino-shi, Tokyo (JP); Tatsuya FUKUI, Musashino-shi, Tokyo (JP); Koji TSUJI, Musashino-shi, Tokyo (JP)

(73) Assignee: NIPPON TELEGRAPH AND TELEPHONE CORPORATION, Tokyo (JP)

(21) Appl. No.: 18/011,448

(22) PCT Filed: Jun. 25, 2020
(86) PCT No.: PCT/JP2020/025004

§ 371 Date: Dec. 19, 2022

(51) Int. Cl. A61B 5/00

(52) U.S. Cl. CPC A61B 5/6801; A61B 5/0015

(57) ABSTRACT

An aspect of the present disclosure is a biosensor information collection system S including: a biosensor terminal 1-N that is to be attached to a living body and outputs sensor information on the living body; a hub terminal 2 that is to be attached to the living body and collects the sensor information on the living body, the biosensor terminal 1-N and the hub terminal 2 performing communication therebetween near a surface of the living body, the hub terminal 2 including: a reception intensity determination unit 26 that determines whether a reception intensity from the biosensor terminal 1-N coincides with a predetermined intensity with an allowable margin or deviates from the predetermined intensity beyond the allowable margin; and a sensor information request unit 25 that requests the sensor information on the living body of the biosensor terminal 1-N when the reception intensity from the biosensor terminal 1-N coincides with the predetermined intensity with the allowable margin and suspends the request for the sensor information on the living body of the biosensor terminal 1-N when the reception intensity from the biosensor terminal 1-N deviates from the predetermined intensity beyond the allowable margin.

### TECHNICAL FIELD

The present disclosure relates to a technology of collecting sensor information on a living body.

### BACKGROUND ART

Technologies of collecting sensor information on a living body are disclosed in Patent Literatures 1 to 5. Examples of the sensor information on the living body include information of the respiratory rate or the cardiac rate of a human or an animal.

### CITATION LIST

### Patent Literature

Patent Literature 1: Japanese Patent No. 6499356

Patent Literature 2: Japanese Patent No. 6444359

Patent Literature 3: Japanese Patent No. 5023088

Patent Literature 4: Japanese Patent Laid-Open No. 2013-055574

Patent Literature 5: Japanese Patent No. 4657125

### SUMMARY OF THE INVENTION

### Technical Problem

In Patent Literatures 1 and 2, the sensor information on a living body is collected using wireless communication between a biosensor terminal and a hub terminal which is distant from the living body. Examples of the wireless communication include Wi-Fi (registered trademark) wireless communication or Bluetooth (registered trademark) wireless communication. There however rises a risk that the sensor information on the living body is intercepted by a separate terminal at meters to tens of meters of distance from the living body. Further, even if the sensor information on the living body is encrypted at the biosensor terminal, this causes some problems from viewpoints of computing resources, transmission bands and power consumption. Furthermore, there rises the frequency of crosstalk and interference of the sensor information on the living body between a plurality of biosensors.

In Patent Literatures 3 to 5, the sensor information on a living body is collected using biological communication between a biosensor terminal and a hub terminal which is attached to the living body. Examples of the biological communication include biological communication using chronological change of an electric field, a current or a magnetic field near the surface of the living body. There can be thereby reduced a risk that the sensor information on the living body is intercepted by a separate terminal at meters to tens of meters of distance from the living body. There however is still high a risk that the sensor information on the living body is intercepted by a separate terminal or a separate living body in contact with the living body. Further, even if the sensor information on the living body is encrypted at the biosensor terminal, this still causes the problems from viewpoints of computing resources, transmission bands and power consumption. Furthermore, there still rises the frequency of crosstalk and interference of the sensor information on the living body between a plurality of biosensors.

Therefore, in order to solve the problems, objects of the present disclosure are, when sensor information on a living body is collected using biological communication between a biosensor terminal and a hub terminal which is attached to the living body, (1) to reduce the risk that the sensor information on the living body is intercepted by a separate terminal or a separate living body in contact with the living body, (2) to eliminate problem(s) from viewpoints of computing resources, transmission bands and power consumption by not performing encryption of the sensor information on the living body at the biosensor terminal, and (3) to reduce the frequency of crosstalk and/or interference of the sensor information on the living body between a plurality of biosensors.

### Means for Solving the Problem

In order to solve the aforementioned problems, there is positively used the fact that an electric field, a current or a magnetic field near the surface of a living body fluctuates since the status of a transmission path near the surface of the living body fluctuates on the occasion when a separate terminal or a separate living body in contact with the living body exists as compared with the occasion when none of a separate terminal or a separate living body in contact with the living body exists. In other words, after the presence or absence of a separate terminal or a separate living body in contact with a living body is sensed based on the presence or absence of the fluctuation of an electric field, a current or a magnetic field near the surface of the living body, it is determined whether or not the sensor information on the living body is to be requested of the biosensor terminal.

Specifically, an aspect of the present disclosure is a biosensor information collection system including: a biosensor terminal that is to be attached to a living body and outputs sensor information on the living body; and a hub terminal that is to be attached to the living body and collects the sensor information on the living body, the biosensor terminal and the hub terminal performing communication therebetween near a surface of the living body, the hub terminal including: a reception intensity determination unit that determines whether a reception intensity from the biosensor terminal coincides with a predetermined intensity with an allowable margin or deviates from the predetermined intensity beyond the allowable margin; and a sensor information request unit that requests the sensor information on the living body of the biosensor terminal when the reception intensity from the biosensor terminal coincides with the predetermined intensity with the allowable margin and suspends a request for the sensor information on the living body with respect to the biosensor terminal when the reception intensity from the biosensor terminal deviates from the predetermined intensity beyond the allowable margin.

Moreover, an aspect of the present disclosure is a biosensor information collection method for collecting sensor information on a living body from a biosensor terminal which is to be attached to the living body via communication near a surface of the living body, the method sequentially including: a reception intensity determination step of determining whether a reception intensity from the biosensor terminal coincides with a predetermined intensity with an allowable margin or deviates from the predetermined intensity beyond the allowable margin; and a sensor information request step of requesting the sensor information on the living body of the biosensor terminal when the reception intensity from the biosensor terminal coincides with the predetermined intensity with the allowable margin and suspending the request for the sensor information on the living body with respect to the biosensor terminal when the reception intensity from the biosensor terminal deviates from the predetermined intensity beyond the allowable margin.

Moreover, an aspect of the present disclosure is a biosensor information collection hub terminal that collects sensor information on a living body from a biosensor terminal which is to be attached to the living body via communication near a surface of the living body, the biosensor information collection hub terminal including: a reception intensity determination unit that determines whether a reception intensity from the biosensor terminal coincides with a predetermined intensity with an allowable margin or deviates from the predetermined intensity beyond the allowable margin; and a sensor information request unit that requests the sensor information on the living body of the biosensor terminal when the reception intensity from the biosensor terminal coincides with the predetermined intensity with the allowable margin and suspends the request for the sensor information on the living body with respect to the biosensor terminal when the reception intensity from the biosensor terminal deviates from the predetermined intensity beyond the allowable margin.

According to these configurations, when the sensor information on the living body is collected using biological communication between the biosensor terminal and the hub terminal which is attached to the living body, (1) the risk that the sensor information on the living body is intercepted by a separate terminal or a separate living body in contact with the living body can be reduced, and (2) problem(s) from viewpoints of computing resources, transmission bands and power consumption can be eliminated by not performing encryption of the sensor information on the living body at the biosensor terminal.

Moreover, an aspect of the present disclosure is the biosensor information collection system, wherein the reception intensity from the biosensor terminal may be a reception intensity of sensor information as a dummy in response to a request for the sensor information as the dummy prior to the request for the sensor information on the living body.

According to this configuration, the sensor information as the dummy only has to be used for measuring the reception intensity from the biosensor terminal without the sensor information on the living body being used, and the risk of interception of the sensor information on the living body by a separate terminal or a separate living body in contact with the living body can be reduced.

Moreover, an aspect of the present disclosure is the biosensor information collection system, wherein the predetermined intensity with respect to the reception intensity from the biosensor terminal may be a reception intensity from the biosensor terminal on the occasion of initial attachment of the biosensor terminal to the living body.

According to this configuration, there can be positively used the fact that the reception intensity from the biosensor terminal fluctuates on the occasion when information that a separate terminal or a separate living body in contact with the living body exists is collected, as compared with the occasion of initial attachment where none of a separate terminal or a separate living body in contact with the living body exists.

Moreover, an aspect of the present disclosure is the biosensor information collection system, wherein the allowable margin with respect to the reception intensity from the biosensor terminal may be set to be narrower/wider as a security level required for communication of the sensor information on the living body is higher/lower.

According to this configuration, there can be set, to a higher level/lower level, the degree of capability of reducing a risk of interception of the sensor information on the living body by a separate terminal or a separate living body in contact with the living body as the security level required for communication of the sensor information on the living body is higher/lower.

Moreover, an aspect of the present disclosure is the biosensor information collection system, wherein the reception intensity determination unit and the sensor information request unit may perform the aforementioned processing when a security level required for communication of the sensor information on the living body is higher than a predetermined level.

According to this configuration, the reception intensity determination processing and the information request determination processing can be caused to be performed/not to be performed, when the security level required for communication of the sensor information on the living body is high/low.

Moreover, an aspect of the present disclosure is the biosensor information collection system, wherein the reception intensity determination unit and the sensor information request unit may perform the aforementioned processing in accordance with priority of collection of the sensor information on the living body, the priority being requested by an external terminal or a user.

According to this configuration, when the sensor information on the living body is collected using biological communication between the biosensor terminal and the hub terminal which is attached to the living body, (3) the frequency of crosstalk and/or interference of the sensor information on the living body between a plurality of biosensors can be reduced, in addition to items (1) and (2).

### Effects of the Invention

As above, according to the present disclosure, when the sensor information on the living body is collected using biological communication between the biosensor terminal and the hub terminal which is attached to the living body, (1) the risk that the sensor information on the living body is intercepted by a separate terminal or a separate living body in contact with the living body can be reduced, (2) problem(s) from viewpoints of computing resources, transmission bands and power consumption can be eliminated by not performing encryption of the sensor information on the living body at the biosensor terminal, and (3) the frequency of crosstalk and/or interference of the sensor information on the living body between a plurality of biosensors can be reduced.

### BRIEF DESCRIPTION OF DRAWINGS

FIG. 1 is a diagram showing a configuration of a biosensor information collection system of the present disclosure.

FIG. 2 is a diagram showing processing of the biosensor information collection hub terminal of the present disclosure.

FIG. 3 is a diagram showing data in a terminal management DB unit of a hub terminal of the present disclosure.

FIG. 4 is a diagram showing collection of biosensor information at a high security level of the present disclosure.

FIG. 5 is a diagram showing collection of biosensor information at a high security level of the present disclosure.

FIG. 6 is a diagram showing collection of biosensor information at a low security level of the present disclosure.

FIG. 7 is a diagram showing collection of biosensor information in accordance with priority of the present disclosure.

### DESCRIPTION OF EMBODIMENTS

Embodiments of the present disclosure will be described with reference to the accompanying drawings. The embodiments described below are examples for carrying out the present disclosure, and the present disclosure is not limited to the embodiments below.

(Configuration of Biosensor Information Collection System of the Present Disclosure)

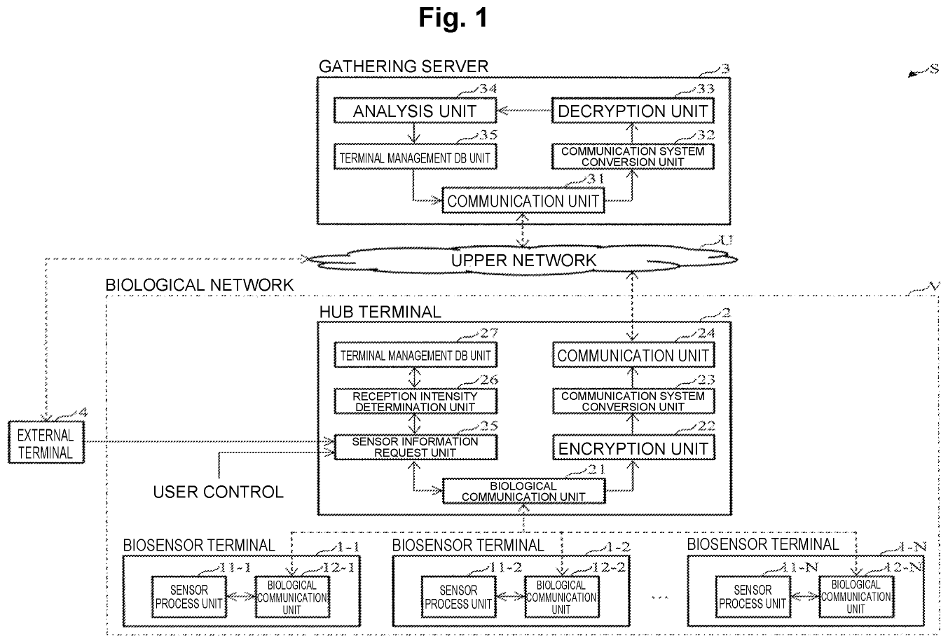

FIG. 1 shows a configuration of a biosensor information collection system of the present disclosure. A biosensor information collection system S includes a biological network V, an upper network U, a gathering server 3 and an external terminal 4. The biological network V is a biological communication network near the surface of a living body. The upper network U is a wired communication network or a wireless communication network. The gathering server 3 includes a communication unit 31, a communication system conversion unit 32, a decryption unit 33, an analysis unit 34 and a terminal management DB unit 35. The external terminal 4 is a mobile terminal or the like which is owned by a human whom biosensor information is to be collected from.

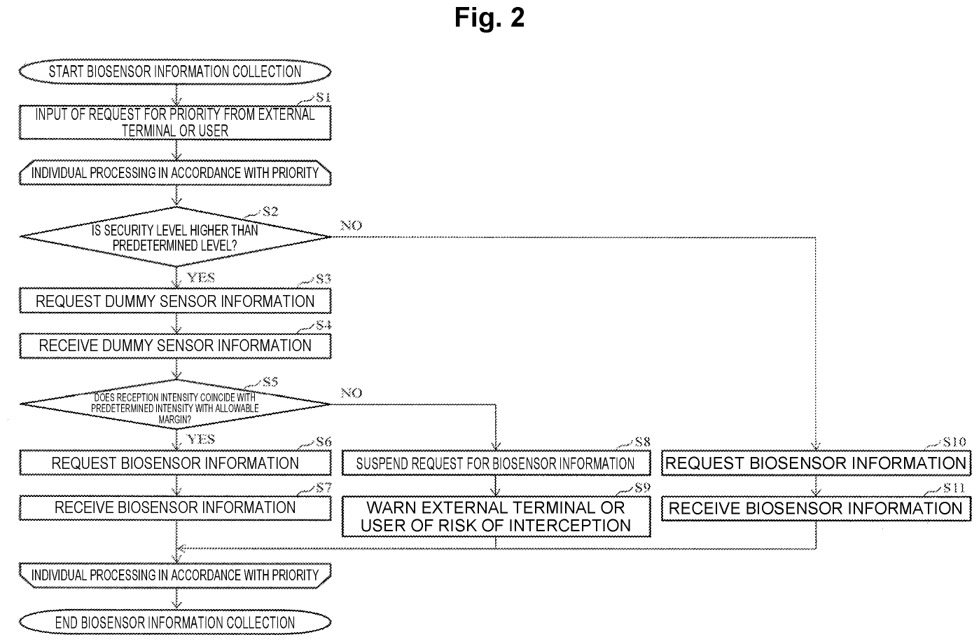

The biological network V includes biosensor terminals 1-1, 1-2, . . . , 1-N and a hub terminal 2 which is attached to the living body. The biosensor terminals 1-1, 1-2, . . . , 1-N include sensor process units 11-1, 11-2, . . . , 11-N and biological communication units 12-1, 12-2, . . . , 12-N, respectively. The hub terminal 2 attached to the living body includes a biological communication unit 21, an encryption unit 22, a communication system conversion unit 23, a communication unit 24, a sensor information request unit 25, a reception intensity determination unit 26 and a terminal management DB unit 27, and can also be implemented by a computer and a biosensor information collection program which is shown in FIG. 2. The biosensor information collection program shown in FIG. 2 can be recorded in a recording medium or can also be provided via a network.

Biological communication between the biosensor terminals 1-1, 1-2, . . . , 1-N and the hub terminal 2 will be described later using FIGS. 2 to 7. The biological communication unit 21 collects sensor information on the living body from the biosensor terminals 1-1, 1-2, . . . , 1-N via the biological network V. The encryption unit 22 encrypts the sensor information on the living body regardless of the presence or absence of encryption at the communication unit 24. The communication system conversion unit 23 converts a communication system into a wired communication system or a wireless communication system. The communication unit 24 transmits the sensor information on the living body to the gathering server 3 via the upper network U.

The communication unit 31 receives the sensor information on the living body from the hub terminal 2 via the upper network U. The communication system conversion unit 32 converts a communication system from a wired communication system or a wireless communication system. The decryption unit 33 decrypts the sensor information on the living body regardless of the presence or absence of encryption at the communication unit 24. The analysis unit 34 analyzes the sensor information on the living body and performs abnormality sensing for the living body. The terminal management DB unit 35 saves information of the sensor analysis on the living body and information of the abnormality sensing for the living body for a fixed period.

(Processing of Biosensor Information Collection Hub Terminal of the Present Disclosure)

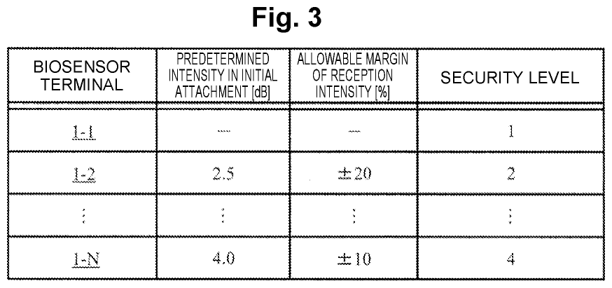

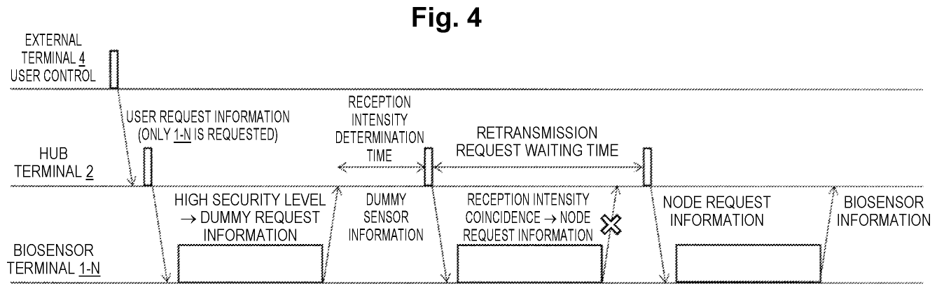

FIG. 2 shows processing of a biosensor information collection hub terminal of the present disclosure. FIG. 3 shows data in a terminal management DB unit of a hub terminal of the present disclosure. FIGS. 4 to 7 show collection of biosensor information of the present disclosure.

In the present disclosure, there is positively used the fact that an electric field, a current or a magnetic field near the surface of a living body fluctuates since the status of a transmission path near the surface of the living body fluctuates on the occasion when a separate terminal or a separate living body in contact with the living body exists as compared with the occasion when none of a separate terminal or a separate living body in contact with the living body exists. In other words, after the presence or absence of a separate terminal or a separate living body in contact with a living body is sensed based on the presence or absence of the fluctuation of an electric field, a current or a magnetic field near the surface of the living body, it is determined whether or not the sensor information on the living body is to be requested of the biosensor terminal 1-N or the like. Hereafter, the present disclosure is described more specifically.

(Collection (1) of Biosensor Information at High Security Level of the Present Disclosure)

FIG. 4 shows collection of biosensor information at a high security level of the present disclosure. The communication unit 31 requests priority of collection of the sensor information on the living body of the external terminal 4 via the upper network U. The external terminal 4 or a user (human whom the biosensor information is to be collected from or the like) requests the priority of collection of the sensor information on the living body to the sensor information request unit 25 (step S1).

Here, as the priority of collection of the sensor information on the living body, only the sensor information on the living body that is to be output from the biosensor terminal 1-N is requested. Further, as to the sensor information on the living body output from the biosensor terminal 1-N, its security level is 4, being high.

The sensor information request unit 25 determines that, as to the sensor information on the living body output from the biosensor terminal 1-N, its security level is 4 with reference to the terminal management DB unit 27, being higher than a predetermined level (for example, 2) (step S2, YES). The sensor information request unit 25 transmits dummy request information to the biosensor terminal 1-N via the biological network V to request sensor information as a dummy of the biosensor terminal 1-N (step S3).

The biological communication unit 12-N receives the dummy request information from the hub terminal 2 via the biological network V. The sensor process unit 11-N outputs the sensor information as the dummy to the biological communication unit 12-N. Examples of the sensor information as the dummy include a beforehand prepared specific pattern or processed sensor information on the living body. The biological communication unit 12-N transmits the sensor information as the dummy to the hub terminal 2 via the biological network V. The biological communication unit 21 receives the sensor information as the dummy from the biosensor terminal 1-N via the biological network V (step S4).

The reception intensity determination unit 26 determines that the reception intensity of the sensor information as the dummy from the biosensor terminal 1-N coincides with a predetermined intensity with an allowable margin with reference to the terminal management DB unit 27 (step S5, YES). Here, the predetermined intensity is 4.0 dB of reception intensity that is from the biosensor terminal 1-N on the occasion of initial attachment of the biosensor terminals 1-1, 1-2, . . . , 1-N to the living body. Further, the allowable margin is ±10%, being a narrow margin, since the security level required for communication of the sensor information on the living body from the biosensor terminal 1-N is 4, being high. The sensor information request unit 25 transmits node request information to the biosensor terminal 1-N via the biological network V to request the sensor information on the living body of the biosensor terminal 1-N (step S6).

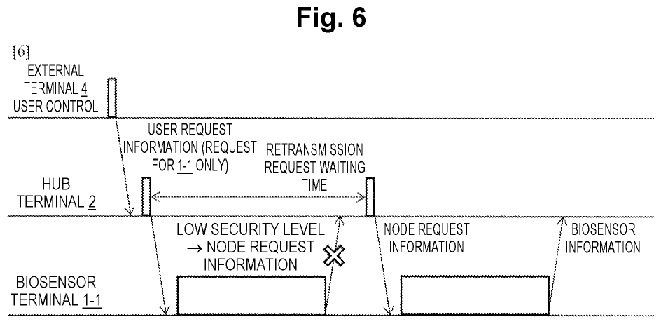

Notably, not being explained with FIG. 4, as to the sensor information on the living body from the biosensor terminal 1-2, the predetermined intensity is 2.5 dB of reception intensity that is from the biosensor terminal 1-2 on the occasion of initial attachment of the biosensor terminals 1-1, 1-2, . . . , 1-N to the living body, and the allowable margin is ±20%, being a wide margin, since the security level required for communication of the sensor information on the living body from the biosensor terminal 1-2 is 2, being low. Moreover, being explained with FIG. 6, as to the sensor information on the living body from the biosensor terminal 1-1, the predetermined intensity and the allowable margin are not particularly set.

The biological communication unit 12-N receives the node request information from the hub terminal 2 via the biological network V. The sensor process unit 11-N outputs the sensor information on the living body to the biological communication unit 12-N. The biological communication unit 12-N transmits the sensor information on the living body to the hub terminal 2 via the biological network V. The biological communication unit 21 receives the sensor information on the living body from the biosensor terminal 1-N via the biological network V (step S7). When the biological communication unit 21 cannot receive the sensor information on the living body from the biosensor terminal 1-N in a retransmission request waiting time, it can also retransmit the node request information to the biosensor terminal 1-N.

In the present disclosure, the reception intensity determination unit 26 determines whether the reception intensity of the sensor information as the “dummy” from the biosensor terminal 1-N coincides with the predetermined intensity with the allowable margin. Then, the sensor information request unit 25 determines whether it starts, “at the initial stage”, to request the sensor information on the living body of the biosensor terminal 1-N. As a modification, the reception intensity determination unit 26 may determine whether the reception intensity of the sensor information on the “living body” from the biosensor terminal 1-N coincides with the predetermined intensity with the allowable margin. Then, the sensor information request unit 25 may determine whether it continues, “in the middle stage”, to request the sensor information on the living body of the biosensor terminal 1-N.

(Collection (2) of Biosensor Information at High Security Level of the Present Disclosure)

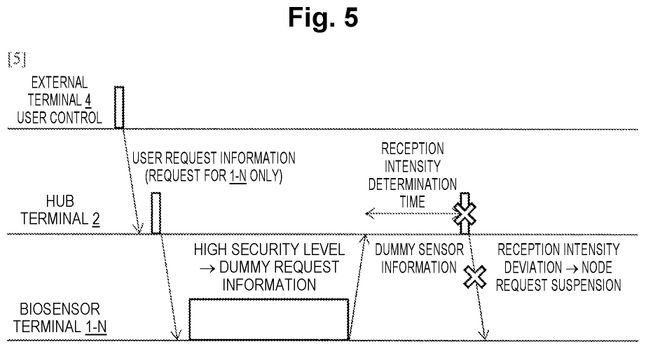

FIG. 5 also shows collection of biosensor information at a high security level of the present disclosure. The communication unit 31 requests the priority of collection of the sensor information on the living body of the external terminal 4 via the upper network U. The external terminal 4 or a user (human whom the biosensor information is to be collected from or the like) requests the priority of collection of the sensor information on the living body of the sensor information request unit 25 (step S1).

Here, as the priority of collection of the sensor information on the living body, only the sensor information on the living body that is to be output from the biosensor terminal 1-N is requested. Further, as to the sensor information on the living body output from the biosensor terminal 1-N, its security level is 4, being high.

The sensor information request unit 25 determines that, as to the sensor information on the living body output from the biosensor terminal 1-N, its security level is 4 with reference to the terminal management DB unit 27, being higher than a predetermined level (for example, 2) (step S2, YES). The sensor information request unit 25 transmits dummy request information to the biosensor terminal 1-N via the biological network V to request sensor information as a dummy of the biosensor terminal 1-N (step S3).

The biological communication unit 12-N receives the dummy request information from the hub terminal 2 via the biological network V. The sensor process unit 11-N outputs the sensor information as the dummy to the biological communication unit 12-N. Examples of the sensor information as the dummy include a beforehand prepared specific pattern or processed sensor information on the living body. The biological communication unit 12-N transmits the sensor information as the dummy to the hub terminal 2 via the biological network V. The biological communication unit 21 receives the sensor information as the dummy from the biosensor terminal 1-N via the biological network V (step S4).

The reception intensity determination unit 26 determines that the reception intensity of the sensor information as the dummy from the biosensor terminal 1-N deviates from a predetermined intensity beyond an allowable margin with reference to the terminal management DB unit 27 (step S5, NO). Here, the predetermined intensity is 4.0 dB of reception intensity that is from the biosensor terminal 1-N on the occasion of initial attachment of the biosensor terminal 1-1, 1-2, . . . , 1-N to the living body. Further, the allowable margin is ±10%, being a narrow margin, since the security level required for communication of the sensor information on the living body from the biosensor terminal 1-N is 4, being high. The sensor information request unit 25 does not transmits node request information to the biosensor terminal 1-N via the biological network V not to request the sensor information on the living body of the biosensor terminal 1-N (step S8).

Notably, not being explained with FIG. 5, as to the sensor information on the living body from the biosensor terminal 1-2, the predetermined intensity is 2.5 dB of reception intensity that is from the biosensor terminal 1-2 on the occasion of initial attachment of the biosensor terminal 1-1, 1-2, . . . , 1-N to the living body, and the allowable margin is ±20%, being a wide margin, since the security level required for communication of the sensor information on the living body from the biosensor terminal 1-2 is 2, being low. Moreover, being explained with FIG. 6, as to the sensor information on the living body from the biosensor terminal 1-1, the predetermined intensity and the allowable margin are not particularly set.

The communication unit 24 warns the external terminal 4 of a risk of interception of the sensor information on the living body via the upper network U (step S9). The sensor information request unit 25 warns the user (human whom the biosensor information is to be collected from or the like) of the risk of interception of the sensor information on the living body (step S9).

(Collection of Biosensor Information at Low Security Level of the Present Disclosure)

FIG. 6 shows collection of biosensor information at a low security level of the present disclosure. The communication unit 31 requests the priority of collection of the sensor information on the living body of the external terminal 4 via the upper network U. The external terminal 4 or a user (human whom the biosensor information is to be collected from or the like) requests the priority of collection of the sensor information on the living body of the sensor information request unit 25 (step S1).

Here, as the priority of collection of the sensor information on the living body, only the sensor information on the living body that is to be output from the biosensor terminal 1-1 is requested. Further, as to the sensor information on the living body output from the biosensor terminal 1-1, its security level is 1, being low.

The sensor information request unit 25 determines that, as to the sensor information on the living body output from the biosensor terminal 1-1, its security level is 1 with reference to the terminal management DB unit 27, being lower than a predetermined level (for example, 2) (step S2, NO). The sensor information request unit 25 transmits node request information to the biosensor terminal 1-1 via the biological network V to request the sensor information on the living body of the biosensor terminal 1-1 (step S10).

The biological communication unit 12-1 receives the node request information from the hub terminal 2 via the biological network V. The sensor process unit 11-1 outputs the sensor information on the living body to the biological communication unit 12-1. The biological communication unit 12-1 transmits the sensor information on the living body to the hub terminal 2 via the biological network V. The biological communication unit 21 receives the sensor information on the living body from the biosensor terminal 1-1 via the biological network V (step S11). When the biological communication unit 21 cannot receive the sensor information on the living body from the biosensor terminal 1-1 in a retransmission request waiting time, it can also retransmit the node request information to the biosensor terminal 1-1.

(Collection of Biosensor Information in Accordance with Priority of the Present Disclosure)

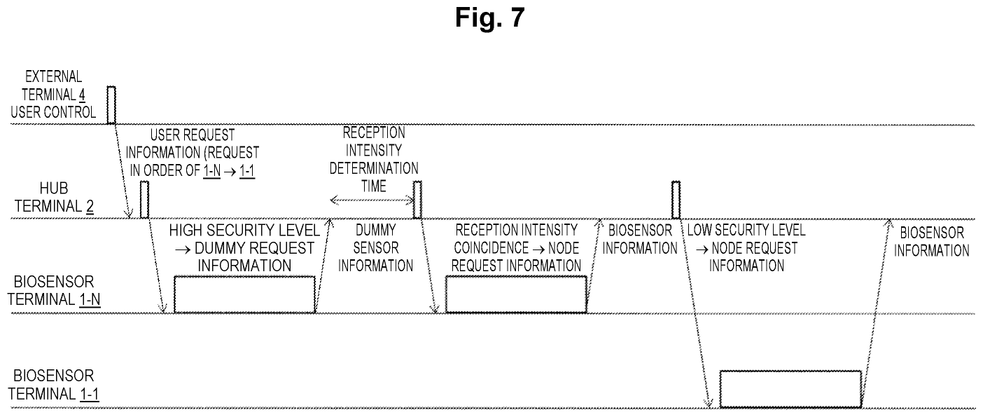

FIG. 7 shows collection of biosensor information in accordance with priority of the present disclosure. The communication unit 31 requests the priority of collection of the sensor information on the living body of the external terminal 4 via the upper network U. The external terminal 4 or a user (human whom the biosensor information is to be collected from or the like) requests the priority of collection of the sensor information on the living body of the sensor information request unit 25 (step S1).

Here, as the priority of collection of the sensor information on the living body, requests are made in the order of the sensor information on the living body output from the biosensor terminal 1-N→the sensor information on the living body output from the biosensor terminal 1-1. Further, as to the sensor information on the living body output from the biosensor terminal 1-N, its security level is 4, being high, and as to the sensor information on the living body output from the biosensor terminal 1-1, its security level is 1, being low.

As to the sensor information on the living body output from the biosensor terminal 1-N, as described with FIGS. 4 and 5, the processing is performed in the order of step S2, YES→steps S3 to S9. As to the sensor information on the living body output from the biosensor terminal 1-1, as described with FIG. 6, the processing is performed in the order of step S2, NO→steps S10 to S11.

(Effects of Biosensor Information Collection System of the Present Disclosure)

When the sensor information on the living body is collected using biological communication between the biosensor terminal 1-N or the like and the hub terminal 2 attached to the living body, (1) a risk of interception of the sensor information on the living body by a separate terminal or a separate living body in contact with the living body can be reduced, and (2) problems from viewpoints of computing resources, transmission bands and power consumption can be eliminated by not performing encryption of the sensor information on the living body at the biosensor terminal 1-N or the like.

Here, the sensor information as the dummy only has to be used for measuring the reception intensity from the biosensor terminal 1-N or the like without the sensor information on the living body being used, and a risk of interception of the sensor information on the living body by a separate terminal or a separate living body in contact with the living body can be reduced.

Further, there can be positively used the fact that the reception intensity from the biosensor terminal 1-N or the like fluctuates on the occasion when information that a separate terminal or a separate living body in contact with the living body exists is collected as compared with the occasion of initial attachment where none of a separate terminal or a separate living body in contact with the living body exists.

Further, there can be set, to a higher level/lower level, the degree of capability of reducing a risk of interception of the sensor information on the living body by a separate terminal or a separate living body in contact with the living body, as the security level required for communication of the sensor information on the living body is higher/lower.

Further, the reception intensity determination processing and the information request determination processing can be caused to be performed/not to be performed, when the security level required for communication of the sensor information on the living body is high/low.

When the sensor information on the living body is collected using biological communication between the biosensor terminal 1-N or the like and the hub terminal 2 attached to the living body, (3) the frequency of crosstalk and/or interference of the sensor information on the living body between a plurality of biosensors 1-N and the like can be reduced, in addition to items (1) and (2).

### INDUSTRIAL APPLICABILITY

With the biosensor information collection system, the biosensor information collection method and the biosensor information collection hub terminal of the present disclosure, information of the respiratory rate or the cardiac rate of a human or an animal or the like can be collected.

### REFERENCE SIGNS LIST

S Biosensor information collection system

V Biological network

U Upper network

1-1, 1-2, 1-N Biosensor terminal

2 Hub terminal

3 Gathering server

4 External terminal

11-1, 11-2, 11-N Sensor process unit

12-1, 12-2, 12-N Biological communication unit

21 Biological communication unit

22 Encryption unit

23 Communication system conversion unit

24 Communication unit

25 Sensor information request unit

26 Reception intensity determination unit

27 Terminal management DB unit

31 Communication unit

32 Communication system conversion unit

33 Decryption unit

34 Analysis unit

35 Terminal management DB unit

1. A biosensor information collection system comprising:
a biosensor terminal that is to be attached to a living body and outputs sensor information on the living body; and
a hub terminal that is to be attached to the living body and collects the sensor information on the living body, the biosensor terminal and the hub terminal performing communication therebetween near a surface of the living body, the hub terminal including:
a reception intensity determination unit that determines whether a reception intensity from the biosensor terminal coincides with a predetermined intensity with an allowable margin or deviates from the predetermined intensity beyond the allowable margin; and
a sensor information request unit that requests the sensor information on the living body of the biosensor terminal when the reception intensity from the biosensor terminal coincides with the predetermined intensity with the allowable margin and suspends the request for the sensor information on the living body with respect to the biosensor terminal when the reception intensity from the biosensor terminal deviates from the predetermined intensity beyond the allowable margin.

2. The biosensor information collection system according to claim 1, wherein the reception intensity from the biosensor terminal is a reception intensity of sensor information as a dummy in response to a request for the sensor information as the dummy prior to the request for the sensor information on the living body.

3. The biosensor information collection system according to claim 1, wherein the predetermined intensity with respect to the reception intensity from the biosensor terminal is a reception intensity from the biosensor terminal on the occasion of initial attachment of the biosensor terminal to the living body.

4. The biosensor information collection system according to claim 1, wherein the allowable margin with respect to the reception intensity from the biosensor terminal is set to be narrower/wider as a security level required for communication of the sensor information on the living body is higher/lower.

5. The biosensor information collection system according to claim 1, wherein the reception intensity determination unit and the sensor information request unit perform the aforementioned processing when a security level required for communication of the sensor information on the living body is higher than a predetermined level.

6. The biosensor information collection system according to claim 1, wherein the reception intensity determination unit and the sensor information request unit perform the aforementioned processing in accordance with priority of collection of the sensor information on the living body, the priority being requested by an external terminal or a user.

7. A biosensor information collection method for collecting sensor information on a living body from a biosensor terminal which is to be attached to the living body via communication near a surface of the living body, the method sequentially comprising:
a reception intensity determination step of determining whether a reception intensity from the biosensor terminal coincides with a predetermined intensity with an allowable margin or deviates from the predetermined intensity beyond the allowable margin; and
a sensor information request step of requesting the sensor information on the living body of the biosensor terminal when the reception intensity from the biosensor terminal coincides with the predetermined intensity with the allowable margin and suspending the request for the sensor information on the living body with respect to the biosensor terminal when the reception intensity from the biosensor terminal deviates from the predetermined intensity beyond the allowable margin.

8. A biosensor information collection hub terminal that collects sensor information on a living body from a biosensor terminal which is to be attached to the living body via communication near a surface of the living body, the biosensor information collection hub terminal comprising:
a reception intensity determination unit that determines whether a reception intensity from the biosensor terminal coincides with a predetermined intensity with an allowable margin or deviates from the predetermined intensity beyond the allowable margin; and
a sensor information request unit that requests the sensor information on the living body of the biosensor terminal when the reception intensity from the biosensor terminal coincides with the predetermined intensity with the allowable margin and suspends the request for the sensor information on the living body with respect to the biosensor terminal when the reception intensity from the biosensor terminal deviates from the predetermined intensity beyond the allowable margin.
